(19) United States
(12) Patent Application Publication
(10) Pub. No.: US 2009/0270437 A1
(43) Pub. Date: Oct. 29, 2009

(54) Use of Buprenorphine or N-Alkylated Derivatives Thereof for Wound Healing

(76) Inventors: Andrew Douglas Baxter, Cambridge (GB); Alan Rothaul, Cambridge (GB)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/514,309

(22) PCT Filed: Nov. 13, 2007
(86) PCT No.: PCT/GB07/04321

§ 371 Date: May 9, 2009

(30) Foreign Application Priority Data
Nov. 14, 2006 (GB) 0622694.8

(51) Int. Cl. A61K 31/4355; A61P 17/02

(52) U.S. Cl. 514/279

(57) ABSTRACT

This invention relates to opioid compounds such as burenorphine or N-alkylated derivatives thereof for wound healing.

### FIELD OF THE INVENTION

This invention relates to the use of opioid compounds in wound care.

### BACKGROUND OF THE INVENTION

Buprenorphine is a complex derivative of the opioid alkaloid thebaine that is a more potent and longer-lasting analgesic than morphine. This atypical opioid analgesic appears to act as a partial agonist at μ and κ opioid receptors while demonstrating antagonist activity at δ receptors. The lack of δ-agonist activity has been suggested to account for the observation that buprenorphine tolerance does not develop with chronic use.

It has been recognized for a while that local analgesia can be achieved with opioid agonists which, when used topically, minimize the CNS side-effects traditionally associated with systemic use. This observation has been used to rationalize the use of opioid creams for the relief of pain in abrasive injuries and burns.

Buprenorphine has poor oral bioavailability and suffers from high first pass metabolism. To avoid this problem, the compound has been delivered by various routes, including parental, intranasal, sub-lingual, buccal, inhaled and transdermal. Buprenorphine has not been used topically but, when delivered transdermally, freely passes through the skin. While buprenorphine suffers from fewer systemic side-effects than morphine, other CNS side-effects such as nausea and emesis are a particular issue when managing the use of this compound.

### SUMMARY OF THE INVENTION

The present invention is based on the discovery that buprenorphine and peripherally acting analogues of buprenorphine are capable of accelerating the healing of both chronic and acute wounds. In addition, buprenorphine and the peripherally acting analogues can be used to boost analgesia when such wounds are associated with pain. Wounds typically associated with pain include chronic dermal wounds, diabetic ulcers, pressure wounds, ophthalmic injury, skin grafts at the graft and donor sites, abrasive wounds and burns.

While opioids applied topically provide some pain relief but are susceptible to causing unwanted systemic and CNS side-effects, these side-effects can be managed by the topical application of buprenorphine and peripherally acting analogues of buprenorphine. For example, controlled delivery of the compound allows retention in the skin and minimizes systemic and ultimately central exposure.

According to the present invention, an inflammatory condition, e.g. as descried above, is treated by the use of a compound of general formula (I):

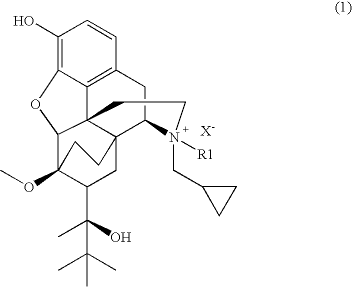

wherein R1 is H, such as in a pharmaceutically acceptable salt of buprenorphine itself, or C₁-C₄ alkyl; and X is counterion of any pharmaceutically acceptable acid, including halide, sulphate, methosulphate, phosphate or of an organic acid such as citrate succinate, tartarate, fumarate, maleate, acetate or methanesulphonate.

### DESCRIPTION OF PREFERRED EMBODIMENTS

Buprenorphine has many chiral centres. Any reference herein to buprenorphine or peripherally acting analogues should be understood as a reference to any enantiomer or mixture thereof. Any enantiomer may be substantially free of others, e.g. in an enantiomeric excess of at least 80%, preferably at least 90% and more preferably at least 95%. Similarly, any mixture of diastereomers may be substantially free of the other. The form drawn above is preferred.

Buprenorphine may be in the form of the free base or any pharmaceutically acceptable salt, as described above. Such forms are known to those of ordinary skill in the art.

The active agent may be administered by the topical dermal, intra-articular or vaginal route. The amount of active ingredient that is used can be chosen by the skilled person having regard to the usual factors.

For use, the active agent is typically formulated, e.g. with conventional diluents or carriers, or as a dressing or a patch, as a medicament adapted to be delivered by the chosen route. Such formulations are designed to retard the passage of the compound into the systemic circulation and are chosen according to such considerations as the potency of the drug, the severity of the condition and the route of administration.

Buprenorphine or a peripherally acting analogue of formula (1) is preferably administered topically to the skin or the eye, percutaneously, intra-articularly or by installation directly into a wound. Indeed, topical delivery introduces significant concentrations of the compound of formula (1) into the skin whilst reducing CNS and peripheral side-effects. In this context, a typical daily dose is less than 50 mg, e.g. 0.1 to 10 mg, buprenorphine; a higher dose, e.g. up to 500 mg of the peripherally acting compounds of formula (1) may be used, especially if transdermal passage is avoided.

Peripherally active compounds of formula (1) can be prepared according to the general scheme below.

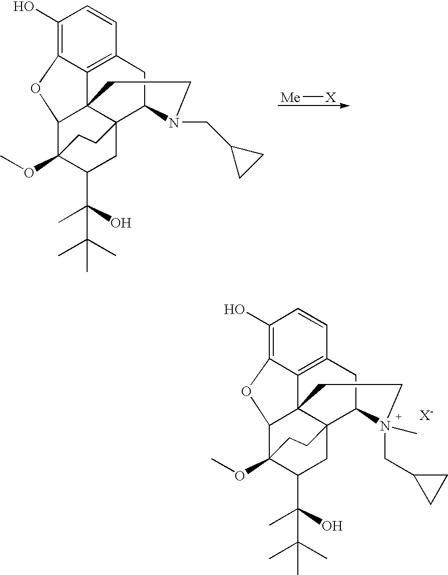

wherein X is typically a reactive halide such as chloride, bromide or iodide, or methosulphate. The transformation may be carried out using methods known to those skilled in the art.

It will often be advantageous to use compounds of formula (I) in combination with another drug used for pain or wound care therapy. Such another drug may be a local anaesthetic, antiseptic or antibiotic. Other combinations include those with an anti-convulsant, such as phenyloin, or a compound used to enhance vascular permeability, including peripheral vasodilators such as adenosine agonists or re-uptake inhibitors (e.g. dipyridamole), anti-platelets agents (e.g. ketanserin) and pentoxyfylline, and vasopeptidase inhibitors such as neutral endopeptidase (NEP) inhibitors or combined ACE/NEP inhibitors.

The following Examples illustrate the invention.

### Buprenorphine Methiodide

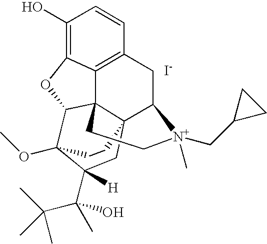

Buprenorphine (5 g, 10.7 mmol) and MeI (34.2 g, 15 ml, 240 mmol) were taken up in MIBK (30 ml), halved between two sealed tubes and heated at 70° C. for six days. The tubes were allowed to cool to RT, combined and evaporated. The residue was triturated with iso-propylacetate, and the solid formed was filtered off and dried. The solid was purified by column chromatography (2%-5% MeOH in CH₂Cl₂). The product obtained was triturated with MTBE and the solid formed was filtered off and dried in vacuo to give 2 g (31% yield) of the title product as a pale yellow solid.

¹H NMR (δ, 400 MHz, d₄-MeOH): 0.50-0.58 (m, 1H), 0.61-0.68 (m, 1H), 0.80-0.95 (m, 2H), 1.025 (s, 9H, ^{t}butyl CH₃), 1.05-1.18 (m, 1H), 1.19-1.3 (m, 2H), 1.40 (s, 3H, CH₃), 1.48-1.68 (m, 2H), 1.85-1.97 (bd, 2H), 2.1 (dd, 1H), 2.45 (dd, 1H), 2.68-2.80 (m, 2H), 3.05 (dd, 1H), 3.15 (dd, 1H), 3.30 (m, 1H), 3.50 (s, 3H, CH₃O), 3.56 (m, 1H), 3.59 (s, 3H, CH₃N), 4.08 (dd, 1H), 4.18 (d, 1H), 4.65 (s, 1H, CH₂CHO), 6.65 (d, 1H, aromatic), 6.74 (d, 1H, aromatic).

### Methodology

A model of ischaemic wound healing was conducted in male rats. Briefly, a 1 cm incisional wound was made on the back of each rat and a subcutaneous pocket was created adjacent to the wound. A 5 mm diameter excisional punch wound was created above this space. Rats were treated topically twice daily with buprenorphine (300 μg/g) or vehicle.

Wound width was measured and wounds were assigned a visual score daily. At the end of the treatment period, the wounds were bisected, and a histological assessment of wound width and re-epithelialisation was performed.

On day 4, wound width was reduced by 29% on average in the drug-treated group, by comparison with vehicle alone (mean for vehicle 3737 mm, and for drug 2680 mm). Also on day 4, wound re-epithelialisation was increased by 29% on average in the drug-treated group, by comparison with vehicle alone (mean for vehicle 55%, and for drug 71.5%).

1. A method for promoting wound healing wherein said method comprises administering, to a patient, buprenorphine or a pharmaceutically acceptable acid addition salt thereof.

2. A method for promoting wound healing, providing analgesia, and/or treating an inflammatory condition, wherein said method comprises administering, to a patient, an analogue of buprenorphine according to formula (1): 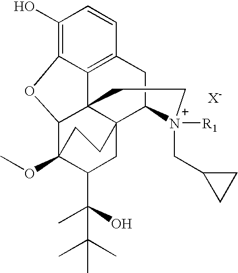
wherein R₁ is C₁-C₄ alkyl and X is a counterion of a pharmaceutically acceptable acid.

3. The method according to claim 2, wherein the counterion is sulphate, methosulphate or phosphate or the acid is citrate, succinate, tartarate, fumarate, maleate, acetate, or methanesulphonate.

4. The method according to claim 1, wherein the salt is a halide.

5. The method according to claim 2, for the treatment of pain experienced by a patient undergoing chronic or acute wound treatment.

6. The method according to claim 1, wherein the wound is a chronic dermal wound, a diabetic ulcer, or is caused by a skin graft.

7. (canceled)

8. (canceled)

9. The method according to claim 6, wherein the wound is on a graft or donor site.

10. The method according to claim 1, wherein the wound is a burn, or is an acute or chronic abrasive injury.

11. (canceled)

12. The method according to claim 1, wherein the administration is via a topical route.

13. The method according to claim 12, wherein the route is percutaneous.

14. The method according to claim 1, which further comprises the administration of a penetration retardant.

15. The method according to claim 1, wherein the administration is via direct installation into a wound.

16. The method according to claim 15, wherein the wound is intra-articular.

17. The method according to claim 1, wherein the wound is ocular.

18. The method according to claim 1, which further comprises administering another agent capable of facilitating or accelerating wound healing.

19. The method according to claim 18, which further comprises administering an anti-convulant and/or a peripheral vasodilator.

20. (canceled)

21. The method according to claim 19, wherein the peripheral vasodilator is an adenosine agonist or reuptake inhibitor.

22. The method according to claim 18, which further comprises administering a vasopeptidase inhibitor.

23. The method according to claim 2 which further comprises administering another agent capable of producing local analgesia.

24. The method according to claim 23, wherein the another agent is lidocaine.

25. The method according to claim 1, wherein the medicament is used in combination with an antiseptic agent and/or a locally delivered antibiotic agent.

26. (canceled)
